(19) United States
(12) Patent Application Publication
(10) Pub. No.: US 2004/0251171 A1
(43) Pub. Date: Dec. 16, 2004

(54) Separation apparatus, method of separation, and process for producing separation apparatus

(76) Inventors: Kazuhiro Iida, Tokyo (JP); Masakazu Baba, Tokyo (JP); Hisao Kawaura, Tokyo (JP); Tohru Sano, Tokyo (JP); Toshitsugu Sakamoto, Tokyo (JP); Noriyuki Iguchi, Tokyo (JP); Hiroko Someya, Tokyo (JP)

Correspondence Address: SUGHRUE MION, PLLC, 2100 PENNSYLVANIA AVENUE, N.W., SUITE 800, WASHINGTON, DC, 20037, US

(21) Appl. No.: 10/495,963

(22) PCT Filed: Nov. 20, 2002
(86) PCT No.: PCT/JP02/12131

(30) Foreign Application Priority Data
Nov. 20, 2001 (JP) 2001-355298

(51) Int. Cl. B07B 1/00; B03B 1/00

(52) U.S. Cl. 209/1

(57) ABSTRACT

There is provided separation techniques for separating samples in a short period of time by using a small amount of samples with excellent resolution, causing few problems such as clogging.

A number of hydrophobic areas 705 are arranged at about equally spaced intervals in a channel where samples pass, and the surface of a hydrophilic substrate 701 is exposed in the area except for the hydrophobic areas 705.

### TECHNICAL FIELD

The present invention relates to a device and a method for separating samples which are different in size, polarity, affinity for water, and the like.

### BACKGROUND ART

For analyzing nucleic acid or protein, samples are often separated and purified in advance, or separated according to their sizes and electric charges. For example, under the Maxam-Gilbert method that is widely used as a base sequencing method, an end of DNA is labeled with ³²P and chemically decomposed so as to obtain fragments of different lengths. After that, the fragments are separated by means of electrophoresis, and a base sequence is decoded by autoradiography. This separating operation is an important factor which determines the length of analyzing time, and it has been a key technical problem in this field to reduce the time taken to complete the separation. In order to solve the problem, it is required to develop a separating device capable of separating desired substances accurately in a short period of time.

Generally, capillary electrophoresis devices have been widely used as such separating device. However, with the use of the capillary electrophoresis device, it takes a long time to carry out measurement, and also a large amount of samples are needed. In addition, a satisfactory level of separative power is not always attained.

### PROBLEMS THAT THE INVENTION IS TO SOLVE

It is therefore an object of the present invention to provide a device and a method for separating desired substances accurately in a short period of time with a small amount of samples.

### DISCLOSURE OF THE INVENTION

In accordance with the present invention, there is provided a separation device comprising: a substrate; and a channel formed on the surface of the substrate for a flow of samples, having a sample feeding part, a sample discharging part, and one or more sample separating parts in between the sample feeding part and sample discharging part; wherein the surface of the sample separating part includes a plurality of first areas two-dimensionally arranged at about equally spaced intervals and second area that occupy the surface of the sample separating part except for the first areas, one being hydrophobic and the other being hydrophilic.

Incidentally, “two-dimensionally arranged at about equally spaced intervals” indicates the state in which the first areas are arranged vertically and horizontally at about equally spaced intervals in an orderly manner.

The separation device may further comprises an external force applying means for moving the samples from the sample feeding part to the sample discharging part by external force. Electric field, surface tension, pressure, etc. can be used as the external force, and examples of the external force applying means include a voltage applying section, a pump and the like. In the case of using surface tension as the external force, there is no need for any particular external force applying means.

The separation device of the present invention may have either configuration:

(i) the first areas are hydrophobic areas and the second area is hydrophilic area; or

(ii) the first areas are hydrophilic areas and the second area is hydrophobic area. Incidentally, in accordance with the present invention, the hydrophilic area has higher hydrophilicity as compared to the hydrophobic area. The level of hydrophilicity can be figured out by measuring the water contact angle.

In the following, the principle of the separation device of the present invention will be described by taking the above case of (i) for example. In this case, a separating target sample is dissolved or dispersed in relatively high hydrophilic solvent, and conducted in the device. Such solvent avoids the surface of the hydrophobic areas (first areas), and distributed only on the hydrophilic area (second area). Consequently, gaps or spaces between the respective hydrophobic area form paths for the target sample, and therefore the time which it takes the sample to pass through the sample separating part is determined depending on the relationship between the space and the size of the sample. Thus, the separation of the sample is carried out according to size.

Besides, in accordance with the present invention, separation is also performed according to the polarity of the sample. Namely, it is possible to separate plural kinds of samples which are different in the level of hydrophilicity/hydrophobicity. In the above-mentioned case of (i), the highly hydrophobic sample is easily caught in the hydrophobic areas, and the time taken to discharge the sample is relatively prolonged. On the other hand, the highly hydrophilic sample is not so easily caught in the hydrophobic areas, and the time taken to discharge the sample is relatively shortened.

As is described above, in accordance with the present invention, separations can be performed according to not only the size of the sample but also its polarity, thus enabling the separation of multicomponent samples, which have heretofore been difficult to separate.

The separation device of the present invention is provided with the sample separating part formed in the surface of the channel as a separating means, differently from the system that carries out separation by barrier structure. Although the size of fine pores in a surface has to be controlled with accuracy for a surface separation, it is often difficult to stably produce a surface having fine pores in desired size and desired form. However, in accordance with the present invention, the sample separating part can be produced by applying surface treating to the channel, and desired separative power can be achieved by adjusting the spaces between the respective first areas. Thus, appropriate device structure can be realized for any purpose without much difficulty. For example, the sample separating part in the separation device of the present invention can be formed by depositing compounds that contain a hydrophobic group on mask openings. The space between the respective hydrophobic areas can be easily controlled by adjusting the width of the mask opening. That is, the space between the hydrophobic areas is properly adjusted depending on the purpose of separation so as to produce the sample separating part that fits the purpose. Particularly, for the separation of protein or DNA, it is required to separate substances of all sizes, from a huge substance to a nano-order one. It has been very difficult to separate such nano-order substance with high resolution in a short period of time by conventional techniques. On the other hand, with the separation device of the present invention, it is possible to separate smaller substances by narrowing the space between the respective first areas. The space between the first areas is achieved through the use of microfabrication technique, and the separation of nano-order-sized substances can be properly implemented.

Moreover, with the separation device of the present invention, separation can be carried out in a short period of time using a small amount of samples. That is, in accordance with the present invention, separation is made based on the surface features of the sample separating part, and accurate separation can be performed. Additionally, there is little loss in samples, and therefore fine separative power can be achieved with a small amount of samples.

Besides, in accordance with the present invention, since separation is made based on the surface features of the channel where samples pass through, the separation device has few clogging problems. Furthermore, the separation device can be cleaned very easily by flushing the surface of the sample separating part with cleaning solution.

In accordance with another aspect of the present invention, there is provided a sample separation method for feeding a sample into the aforementioned separation device from its sample feeding part and separating prescribed components from the sample.

With the sample separation method, a highly accurate sample separation can be realized while resolving such problems as clogging.

In accordance with yet another aspect of the present invention, there is provided a method for manufacturing a separation device comprising a substrate having a hydrophilic surface, a channel formed on the surface of the substrate for a flow of samples, and sample separating parts provided to the channel, comprising the steps of:

cutting a groove on the surface of the substrate to form the channel; and

after making a mask having openings on at least a part of the surface of the channel, depositing a compound that contains a hydrophobic group on the surface of the channel from the openings, and removing the mask so as to form respective sample separating parts each including a plurality of hydrophobic areas arranged two-dimensionally at about equally spaced intervals.

In accordance with yet a further aspect of the present invention, there is provided a method for manufacturing a separation device comprising a substrate having a hydrophobic surface, a channel formed on the surface of the substrate for a flow of samples, and sample separating parts provided to the channel, comprising the steps of:

cutting a groove on the surface of the substrate to form the channel; and

after making a mask having openings on at least a part of the surface of the channel, depositing a compound that contains a hydrophilic group on the surface of the channel from the openings, and removing the mask so as to form the respective sample separating parts each including a plurality of hydrophobic areas arranged two-dimensionally at about equally spaced intervals.

For example, a silane coupling agent can be used as the compound that contains a hydrophobic/hydrophilic group.

With the manufacturing method of the present invention, it is possible to produce patterns for a mixture of hydrophobic areas and hydrophilic areas with a high degree of accuracy in a good yield.

### BRIEF DESCRIPTION OF THE DRAWINGS

FIG. 1 is a diagram showing an example of a separation device according to the present invention;

FIG. 2 is a diagram showing the detailed configuration of a separating channel depicted in FIG. 1;

FIG. 3 is a couple of diagrams showing the detailed configuration of a separating channel depicted in FIG. 1;

FIG. 4 is a diagram for explaining a method for separating a sample;

FIG. 5 is a diagram for explaining a method for separating a sample;

FIG. 6 is a diagram illustrating a method for applying a correction voltage to adjust electro-osmosis flow;

FIG. 7 is a plan view schematically showing the configuration of a separation device according to the present invention;

FIG. 8 is a series of cross section diagrams for explaining a process for manufacturing a separation device according to the present invention;

FIG. 9 is a series of cross section diagrams for explaining a process for manufacturing a separation device according to the present invention;

FIG. 10 is a series of cross section diagrams for explaining a process for manufacturing a separation device according to the present invention;

FIG. 11 is a couple of cross section diagrams for explaining a process for manufacturing a separation device according to the present invention;

FIG. 12 is a couple of cross section diagrams for explaining a process for manufacturing a separation device according to the present invention;

FIG. 13 is a plan view schematically showing the configuration of a separation device according to the present invention;

FIG. 14 is a couple of diagrams for explaining a method for manufacturing a separation device according to the present invention;

FIG. 15 is a cross section diagram schematically showing the configuration of a separation device according to the present invention;

FIG. 16 is a schematic picture of a microgram showing bubbles formed on a hydrophobic patch; and

FIG. 17 is a schematic picture of a microgram showing beads in collision with bubbles.

Incidentally, the numerals 101a and 101b represent reservoirs. The numerals 102a and 102b represent reservoirs. The numerals 103a and 103b represent reservoirs. The numeral 110 represents a substrate. The numeral 111 represents an sample injection channel. The numeral 112 represents a separating channel. The numeral 113 represent a detector. The numeral 114 represents a collecting channel. The numeral 701 represents a substrate. The numeral 702 represents an electron beam exposure resist. The numeral 702a represents an unexposed part. The numeral 702b represents an exposed part. The numeral 703 represents a hydrophilic area. The numeral 705 represents a hydrophobic area. The numeral 706 represents a sample separating part. The numeral 710 represents a hard mask. The numeral 711 represents a resist mask. The numeral 720 represents a hydrophobic surface treated layer. The numeral 721 represents a resist. The numeral 730 represents a groove. The numeral 731 represents a sample separating area. The numeral 902 represents a glass substrate. The numeral 903 represents a hydrophobic layer. The numeral 904 represents a space.

### BEST MODE FOR CARRYING OUT THE INVENTION

In accordance with the present invention, the sample separating part can be produced in such a manner as to providing hydrophobic treatment to parts of the hydrophilic surface of the substrate, or providing hydrophilic treatment to parts of the hydrophobic surface of the substrate. Alternatively, the sample separating part can be produced by giving both hydrophobic and hydrophilic treatments to the surface of the substrate.

Examples of the substrate having a hydrophilic surface include a quartz substrate and a glass substrate. On the other hand, examples of the substrate having a hydrophobic surface include resin substrates such as a silicone resin substrate and a polyethylene resin substrate.

In order to implement the hydrophobic/hydrophilic treatment, for example, a compound consisting of a unit which is adsorbed by or chemically bonded with substrate material and a unit having a hydrophobic/hydrophilic accessory group is fixed or immobilized on or coupled with the surface of the substrate. A silane coupling agent, etc. can be used as such compound.

Examples of the silane coupling agent include:

Vinyltrichlorosilane;

Vinyltrimethoxysilane;

Vinyltriethoxysilane;

β-(3,4-epoxycyclohexyl) ethyltrimethoxysilane;

γ-glycidoxypropyltrimethoxysilane;

γ-glycidoxypropylmethyldiethoxysilane;

γ-glycidoxypropyltriethoxysilane;

γ-methacryloxypropylmethyldimethoxysilane;

γ-methacryloxypropyltrimethoxysilane;

γ-methacryloxypropylmethyldiethoxysilane;

γ-methacryloxypropyltriethoxysilane;

N-β(aminoethyl)-γ-aminopropylmethyldimethoxysilane;

N-β(aminoethyl)-γ-aminopropyltrimethoxysilane;

N-β(aminoethyl)-γ-aminopropyltriethoxysilane;

γ-aminopropyltrimethoxysilane;

γ-aminopropyltriethoxysilane;

N-phenyl-γ-aminopropyltrimethoxysilane;

γ-chloropropyltrimethoxysilane;

γ-mercaptopropyltrimethoxysilane;

3-isocyanatepropyltriethoxysilane;

3-acryloxypropyltrimethoxysilane;

3-triethoxysilyl-N-(1,3-dimethyl-butylidene); and

3-thiolpropyltriethoxysilane.

Among them, those containing a amino group such as N-β (aminoethyl)-γ-aminopropylmethyldimethoxysilane, N-β(aminoethyl)-γ-aminopropyltrimethoxysilane, N-β(aminoethyl)-γ-aminopropyltriethoxysilane, γ-aminopropyltrimethoxysilane, γ-aminopropyltriethoxysilane, N-phenyl -γ-aminopropyltrimethoxysilane, etc. are the preferred silane coupling agent that contains a hydrophilic group.

On the other hand, those containing a thiol group such as 3-thiolpropyltriethoxysilane and the like are the preferred silane coupling agent that contains a hydrophobic group.

For applying the coupling agent, there are utilized the spin coat method, spray method, dip method, vapor-phase method and the like. Under the spin coat method, the coupling agent or a liquid in which constituents of a bounding layer are dissolved or dispersed is applied by a spin coater. According to this method, excellent control of layer thickness can be achieved. Under the spray method, the coupling agent is sprayed over the substrate and, under the dip method, the substrate is dipped in the coupling agent. By these methods, a layer can be formed from a simple process without using special equipment. Besides, under the vapor-phase method, the substrate is heated if necessary, and vapors of the coupling agent, etc. are floated thereon. According to this method, a fine layer can also be formed with excellent thickness control. Especially, spin-coating of a silane coupling agent solution is preferably used since excellent adhesion can be stably achieved. On this occasion, the preferable range of the concentration of the silane coupling agent solution is from 0.01 to 5 v/v %, more desirably from 0.05 to 1 v/v %. As a solvent for the silane coupling agent solution, purified water, alcohol such as methanol, ethanol, isopropyl alcohol, and ester such as ethyl acetate can be used either singly or in mixtures. Among them, methanol, ethanol or ethyl acetate diluted with purified water is preferably used because a marked improvement is shown in adhesion. After applying the coupling agent to the substrate, it is dried. While there is no special limitation on the drying temperature, the drying is normally performed at temperatures ranging from room temperature (25° C.) to 170° C. Drying time ranges, although depending on the drying temperature, from 0.5 to 24 hours. The drying may be carried out in the air or in the inert gas such as nitrogen. For example, the coupling agent can be dried by blowing nitrogen against the substrate.

In accordance with the present invention, the first area is not especially limited in shape, and may have a round, oval, square, triangular, etc. shape. The first area may also be a convexity of prescribed height through the hydrophobic surface treatment. In addition, there is no special limitation on the size of the first area, and the size is decided according to the intended purpose and the application of the separation device.

The separation device of the present invention can be used for separating/purifying fluid samples which are different in size, polarity and the like. In particular, the separation device is suitable for the separation of biological material. For example, with the use of human or other animal saliva as a sample, the separation device is suitably used for separating/concentrating components as follows:

(i) separation/concentration of cells and other constituents;

(ii) separation/concentration of solid materials (fragments of cell membranes, mitochondria, endoplasmic reticula) and liquid fractions (cytoplasm) in the components obtained by homogenizing a cell;

(iii) separation/concentration of high-molecular-weight components (DNA, RNA, protein, sugar chains) and low-molecular-weight components (steroid, glucose, etc.) in the components of the liquid fractions; and

(iv) separation of polymer lysates.

The separation device of the present invention is capable of separating microscopic substances, and applicable to the separation/purification of nucleic acid fragments in various sizes, organic molecules of amino acid, peptide, protein, etc., metal ions and the like.

In accordance with the present invention, the space between the respective first areas is decided according to the purpose of separation. For example, in such processes as:

(i) separation/concentration of cells and other constituents;

(ii) separation/concentration of solid materials (fragments of cell membranes, mitochondria, endoplasmic reticula) and liquid fractions (cytoplasm) in the components obtained by homogenizing a cell; and

(iii) separation/concentration of high-molecular-weight components (DNA, RNA, protein, sugar chains) and low-molecular-weight components (steroid, glucose, etc.) in the components of the liquid fractions; the space is respectively set to:

(i) 1 μm to 10 μm;

(i) 100 nm to 1 μm; and

(i) 1 nm to 100 nm.

The separation device of the present invention serves as an analytical device when provided with a detecting section on the downstream side of its sample separating part. Besides, the separation device may be configured so that prescribed components can be taken out from the sample discharging part.

As is described above, the space between the respective first areas is decided according to the purpose of separation. The space can be set to, for example, less than 100 nm. Since the hydrophobic area can be produced by deposition process based on lithographic techniques such as electron beam exposure, the space of less than 100 nm, and further, the space of less than 50 nm can be obtained. Consequently, it is possible to separate the components that have heretofore been difficult to separate.

The separation device of the present invention may include a plurality of sample separating parts and paths through the sample separating parts for samples to pass. With this construction, samples are separated based on the principle other than the general molecular sieve. While the separation device of the present invention is capable of the separation according to the size, hydrophilicity/hydrophobicity, affinity for water, and polarity of samples, the separation by sizes will be specifically explained below.

By the general molecular sieve, substances having larger molecules are more severely prevented from passing through the sieve. Consequently, the separation is made in such a manner that a larger substance is eluted after smaller one has been eluted. On the other hand, in accordance with the present invention, a smaller sample travels a longer distance in the sample separating part, and therefore the separation is made in such a manner that a smaller substance is eluted after larger one has been eluted. In other words, large-sized substances pass through the separating area relatively smoothly. As a result, throughput in the separating operation is considerably enhanced. Especially, in the case of separating nucleic acid, protein or the like, the radius of gyration of molecules exhibits a wide range of variation, and large-sized substances are likely to reduce separation efficiency. The present invention solves this problem, and can be suitably applied to the separation of nucleic acid, protein or the like.

Incidentally, the width of the paths through the sample separating parts may be formed wider than an average space between the respective hydrophobic areas in the sample separating part. With this construction, a large-sized substance smoothly passes through the path among the sample separating parts, and also a small-sized substance passes through the sample separating parts after having traveled a certain distance according to its size. Consequently, the separation by which a larger substance is eluted after smaller one has been eluted is also smoothly performed.

Besides, a space between the respective first areas in each sample separating part can be set to arbitrary distance with respect to each sample separating part. Therefore, in accordance with the present invention, it is possible to arbitrarily set two types of parameters, that is, the distance between the respective first areas in each sample separating part and the width of the path through the sample separating parts, thus enabling the separation of samples in a large variety of sizes with high resolution without the occurrence of clogging and the deterioration of throughput. For example, to separate small-sized molecules with high resolution, clogging and the deterioration of separation efficiency can be prevented by narrowing the space between the first areas to the order of several nanometers to several dozen nanometers and, at the same time, widening the path through the sample separating parts so that large-sized molecules move smoothly.

The first areas included in the sample separating part may be of substantially the same size and equally spaced. By this means, the sensitivity of separation can be heightened. When the sample separating part includes more of the first areas, its resolution is enhanced.

The sample separating part may be comprised of the first areas in different sizes. That is, the first areas may be formed in different sizes and arranged at different intervals. By this means, it is possible to separate samples of a large variety of sizes with high resolution without the occurrence of clogging and the deterioration of throughput.

The separation device of the present invention may further comprises an external force applying means for moving samples in the channel by external force. With this construction, the accuracy of separation and the time required for separation can be set properly for any purpose by controlling the level of applied external force. As the external force, it is preferable to use pressure or electric field because those forces do not require any special external force applying member. Besides, samples can be moved by using the capillary phenomenon. In this case, there is no need for the external force applying means, thus enabling the miniaturization of the device.

For example, the separation device of the present invention is used for the separation of samples as follows:

(i) separation/concentration of cells and other constituents;

(ii) separation/concentration of solid materials (fragments of cell membranes, mitochondria, endoplasmic reticula) and liquid fractions (cytoplasm) in the components obtained by homogenizing a cell;

(iii) separation/concentration of high-molecular-weight components (DNA, RNA, protein, sugar chains) and low-molecular-weight components (steroid, glucose, etc.) in the components of the liquid fractions; and

(iv) separation of polymer lysates.

Examples of microscopic sample include nucleic acid or nucleic acid fragments, organic molecules of amino acid, peptide, protein, etc., metal ions and the like. Particularly, the separation device is effective when using nucleic acid or protein as a sample. For separating these samples, it is necessary to separate small-sized molecules with high resolution, and therefore the separation device has to be provided with minute spaces on the order of several nanometers to several dozen nanometers. At the same time, it is required to efficiently restrain clogging caused by a large substance. The separation device of the present invention can handle both the requirements, and is suitable for the separation of nucleic acid or protein.

In the above-mentioned separation device, the sample separating part formed over the surface of the channel may be divided by slits into plural parts. With this construction, a band in the detecting section takes a linear shape, and the detecting area can be broaden, thus improving detection sensitivity.

Incidentally, the separation device of the present invention just requires the inclusion of the sample separating part, and not necessarily includes the sample feeding part and external force applying means therein. For example, the separation device of the present invention may be of throwaway cartridge type, and incorporated in a prescribed unit in use.

Referring now to the drawings, a description of preferred embodiments of the present invention will be given in detail.

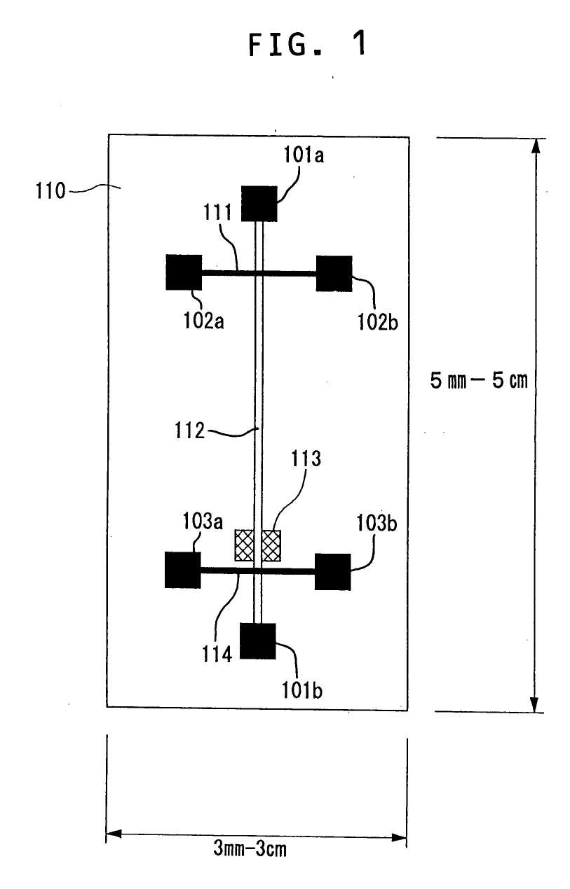

FIG. 1 is a diagram showing an example of the separation device according to the present invention. In FIG. 1, a separating channel 112 is formed on a substrate 110. An sample injection channel 111 and a collecting channel 114 are formed so as to cross the separating channel 112. Reservoirs 101(a, b), 102(a, b), and 103(a, b) are formed at the both ends of the sample injection channel 111, separating channel 112 and collecting channel 114, respectively. The separating channel 112 is provided with a detector 113. While appropriate values are selected according to application for the outside dimension of the separation device, the values are generally set to 5 mm to 5 cm by 3 mm to 3 cm. The sample separating part is formed in a part of the separating channel 112. The position of the sample separating part is properly set in consideration of separation efficiency and the like. For example, when forming the sample separating part in the vicinity of the intersection of the sample injection channel 111 and separating channel 112 on the downstream side of the sample injection channel 111, the separation of samples is carried out efficiently.

Next, a description will be given of a method for performing the separation of samples with the separation device. In this embodiment, a sample for separation is dissolved or dispersed in purified water, a mixture of purified water and hydrophilic solvent, or carrier solvent such as buffer solution to be used. Examples of preferred carrier solvent include a mixture of water and isopropyl alcohol, trimethylammonium, aqueous solution including boric acid and ethylenediamine tetra acetic acid (EDTA), and aqueous sodium phosphate solution.

In preparing for separation, each channel in the separation device is filled with a carrier solvent. Subsequently, a sample is fed in the reservoir 102a or 102b. When feeding the sample in the reservoir 102a, a voltage is applied so that the sample flows toward the reservoir 102b. On the other hand, when feeding the sample in the reservoir 102b, a voltage is applied so that the sample flows toward the reservoir 102a. Accordingly, the sample flows into the sample injection channel 111 to fill the entire sample injection channel 111. At this point, the sample in the separating channel 112 is present only at the intersection with the sample injection channel 111, and forms a band about the width of the sample injection channel 111.

After that, having stopped applying a voltage across the reservoirs 102a and 102b, a voltage is applied between the reservoirs 101a and 101b so that the sample flows toward the reservoir 101b. Hereby, the sample passes through the separating channel 112 at a speed according to the size of molecules, the strength of charges, and the size of the space between the respective first areas. Consequently, different molecule groups in the sample are separated into bands moving at different speeds. When the separated bands reach the detector 113, the detector 113 detects the bands by an optical or physico-chemical method. Optical detection is performed, for example, by irradiating molecules to which fluorescent material is attached with a laser at the detector 113, and observing fluorescence emitted from the molecules. The separated bands can be collected with respect to each band. When applying a voltage across the reservoirs 103a and 103b after having stopped applying a voltage across the reservoirs 101a and 101b in timing with the passing of a desired band through the detector 113, bands that exist at the intersection of the separating channel 112 and collecting channel 114 flow into the collecting channel 114. When the application of voltage across the reservoirs 103a and 103b is stopped after a prescribed period of time, desired molecules in the separated band can be obtained.

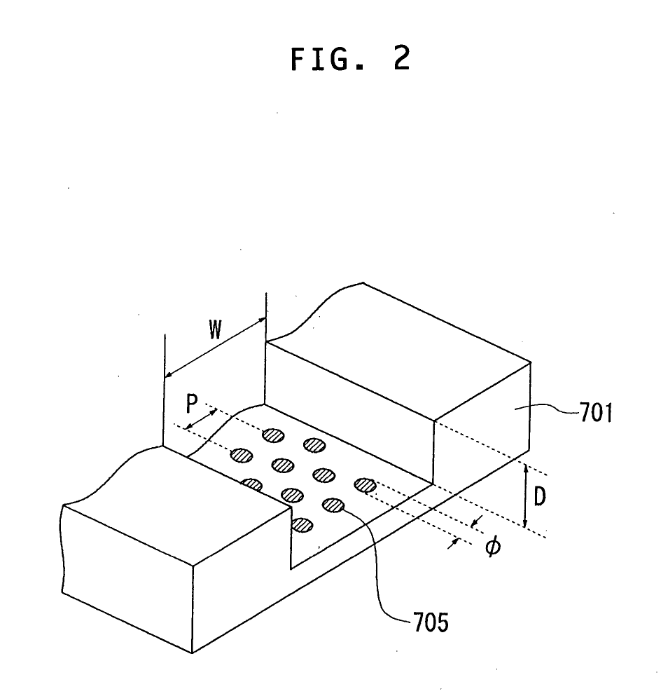

In the following, a description will be given of the configuration of the separating channel in the separation device. FIG. 2 is a diagram showing the detailed configuration of the separating channel 112 depicted in FIG. 1. In FIG. 2, a groove having a depth of D is formed on a substrate 701, and hydrophobic areas 705 each having a diameter of Φ are formed regularly at equally spaced intervals in the groove. In this embodiment, a coupling agent that contains a hydrophobic group is fixed or immobilized on or coupled with the surface of the substrate 701 to form the hydrophobic areas 705. Although not shown in FIG. 2, the channel is generally provided with a cover thereon, thus preventing the evaporation of solvent and enabling a sample in the channel to move by pressure. Incidentally, the separation device may not include such cover.

In FIG. 2, the dimension of each part is set as follows:

W: 10 to 20 μm;

W: 50 nm to 10 μm;

φ: 10 to 1000 nm;

d: 10 nm to 100 μm; and

p: 50 nm to 10 μm.

The dimension is decided according to the purpose of separation. For example, concerning p in such processes as:

(i) separation/concentration of cells and other constituents;

(ii) separation/concentration of solid materials (fragments of cell membranes, mitochondria, endoplasmic reticula) and liquid fractions (cytoplasm) in the components obtained by homogenizing a cell; and

(iii) separation/concentration of high-molecular-weight components (DNA, RNA, protein, sugar chains) and low-molecular-weight components (steroid, glucose, etc.) in the components of the liquid fractions; the value is respectively set to:

(i) 1 μm to 10 μm;

(i) 100 nm to 1 μm; and

(i) 1 nm to 100 nm.

Besides, the value of depth D is such an important factor as to affect separative power, and preferably set to 1 to 10 times the radius of gyration of a separating target sample, more desirably 1 to 5 times the radius of gyration of a sample.

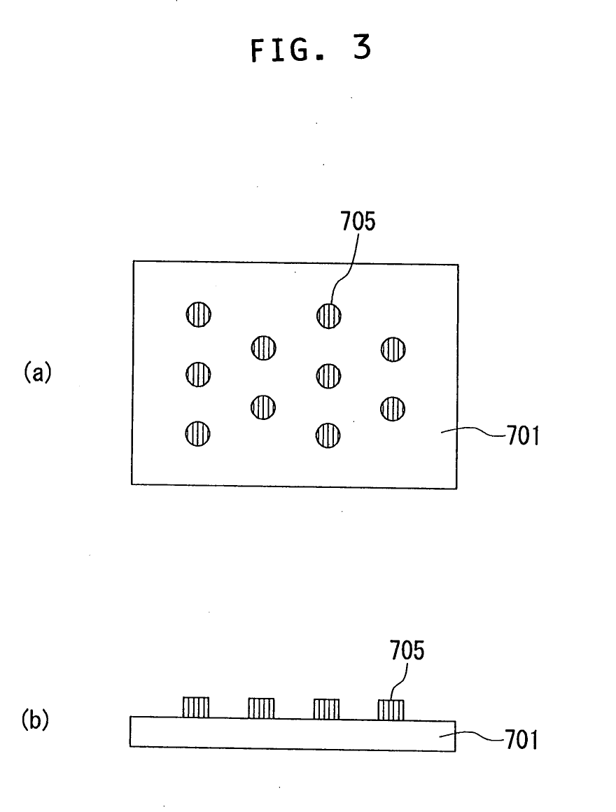

FIG. 3 is a couple of diagrams showing the overhead view (FIG. 3(a)) and side view (FIG. 3(b)) of the separating channel depicted in FIG. 2. Each of the hydrophobic areas 705 is generally 0.1 to 100 nm in layer thickness. In part other than the hydrophobic areas 705, the surface of the substrate 701 is exposed. With the use of hydrophilic material such as glass for the substrate 701 shown in FIG. 2, hydrophobic areas are formed in a prescribed pattern on its hydrophilic surface, thus implementing sample separating functions. That is, when using the above-mentioned hydrophilic buffer solution, etc. as carrier solvent, samples pass on the hydrophilic surface only, but not on the hydrophobic areas. Consequently, the hydrophobic areas 705 serve as obstacles to the flow of samples, which brings about sample separating functions.

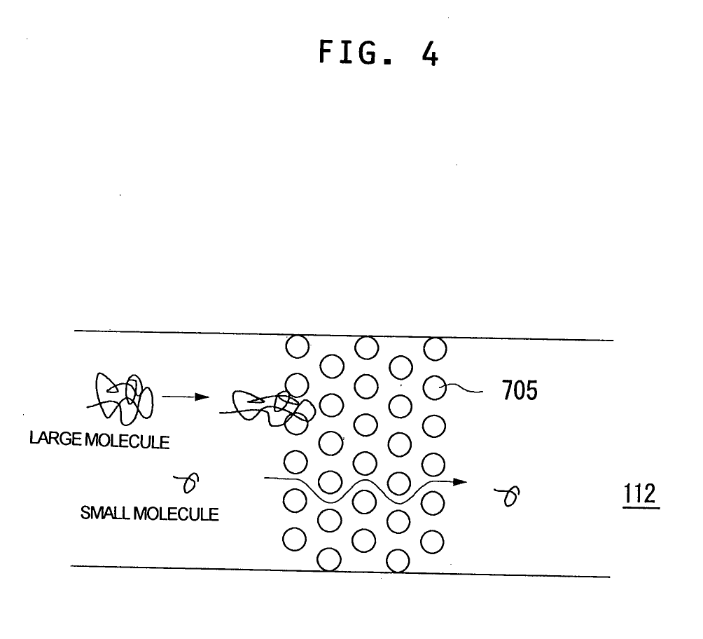

Next, a description will be given of separation methods or styles depending on the patterns of the hydrophobic areas 705 in terms of the size of molecules. There are two conceivable styles for separation. One of them is illustrated in FIG. 4. In this style, the larger molecules are, the bigger obstruction the hydrophobic areas 705 cause, and it takes large molecules a long time to path through the separating area shown in FIG. 4. On the other hand, small molecules pass the spaces between the respective hydrophobic areas 705 relatively smoothly, and pass through the separating area in a shorter period of time as compared with large molecules.

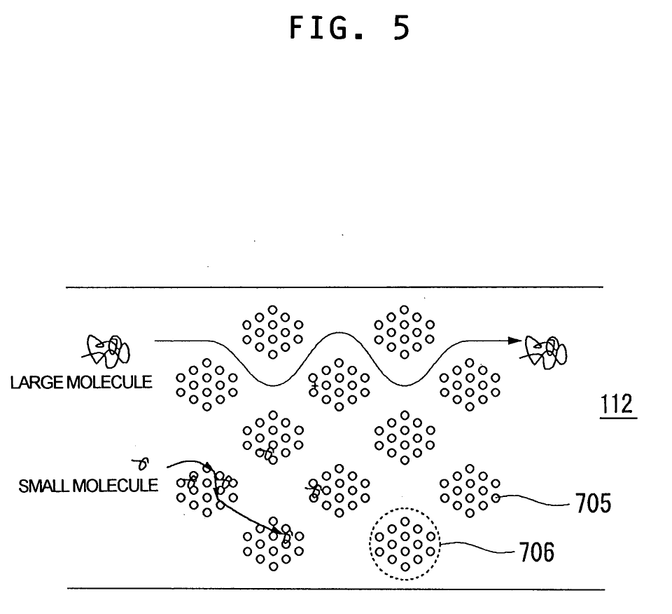

In the other style shown in FIG. 5, large molecules flow out swiftly and small molecules flow out slowly on the contrary to the case of FIG. 4. In the style of FIG. 4, when a large-sized substance is included in a sample, the substance sometimes blocks up the space between the hydrophobic areas 705, resulting in the deterioration of separation efficiency. The separation style shown in FIG. 5 solves such problem. In FIG. 5, a plurality of sample separating parts 706 are formed at spaced intervals in the separating channel 112. In the respective separating parts 706, the hydrophobic areas 705 of a similar size are arranged at equally spaced intervals.

Since there are formed among the sample separating parts 706 paths wide enough for large molecules to pass, large molecules flow out swiftly and small molecules flow out slowly on the contrary to the case of FIG. 4. That is, smaller molecules are more likely to get trapped, and travel a longer distance in the separating area. On the other hand, large molecules pass through the paths among the sample separating parts 706 smoothly. Consequently, the separation is made in such a manner that a smaller substance is eluted after larger one has been eluted. Since large molecules pass through the separating area relatively smoothly, the aforementioned clogging problem caused by trapped large molecules can be reduced, thus achieving a significant improvement in separation efficiency. In order to produce a better effect, it is preferable that the path through the sample separating parts 706 is wider than the space between two adjacent hydrophobic areas 705. The width of the path is preferably set to 2 to 200 times the space between the hydrophobic areas 705, more desirably 5 to 100 times the space.

Incidentally, while the hydrophobic areas 705 of the same size are arranged at equally spaced intervals in the respective sample separating parts in the case of FIG. 5, the sample separating parts may be comprised of the hydrophobic areas 705 in different sizes being arranged at different intervals.

When separating a molecule-sized substance, the width of the path through the sample separating parts and the distance between the adjacent first areas in each sample separating part are properly determined according to the size of separating target components (organic molecules of nucleic acid, amino acid, peptide, protein, etc., and molecules/ions of chelated metal). For example, the space between two adjacent first areas is preferably set to be the same as or only slightly smaller/greater than the radius of gyration of the smallest molecule. More specifically, the space between the first areas is set so that the difference between the sizes of the radius of gyration of the smallest molecule and the space is within 100 nm, preferably within 50 nm, and more desirably within 10 nm. Separative power can be further improved by appropriately setting the space between the respective first areas.

The distance between adjacent sample separating parts (the width of the path) is preferably set to be the same as or only slightly smaller/greater than the radius of gyration of the largest molecule. More specifically, the distance between adjacent sample separating parts is set so that the difference between the radius of gyration of the largest molecule and the distance is within 10% of the radius of gyration of the largest molecule, preferably within 5% thereof, and more desirably within 1% thereof. When the distance between adjacent sample separating parts is too wide, the separation of small molecules sometimes shows unsatisfactory results. On the other hand, when the distance between adjacent sample separating parts is too narrow, clogging is more likely to occur.

Besides, while the hydrophobic areas are arranged at equally spaced intervals in the aforementioned embodiment, the hydrophobic areas may be arranged at different intervals in the respective sample separating parts. By this means, large, medium and small molecules/ions in various sizes can be separated efficiently. In addition, it is also available to arrange the hydrophobic areas alternately in a direction of movement of samples. With this construction, target component can be separated efficiently.

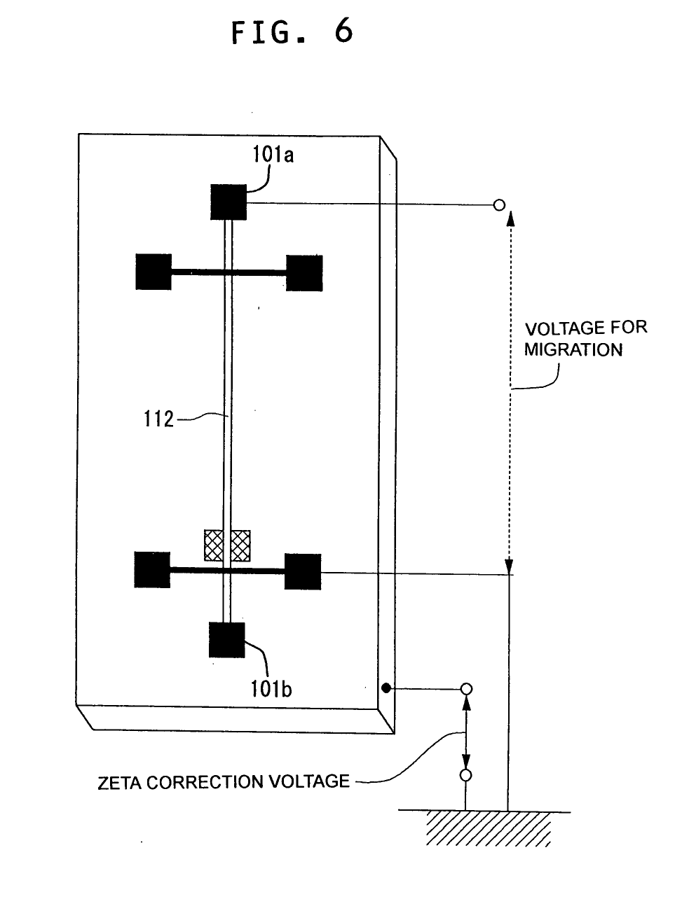

In the separation device of the present invention, a voltage is applied to both ends of the separating channel 112 so as to move samples therein as shown in FIG. 6. At this point, a voltage for suppressing electro-osmosis flow may be applied in addition to the voltage for moving samples by external force. In the case of FIG. 6, a zeta correction voltage is applied to the substrate for that purpose. By this means, electro-osmosis flow can be suppressed, and it is possible to effectively prevent the broadening of the measurement peak.

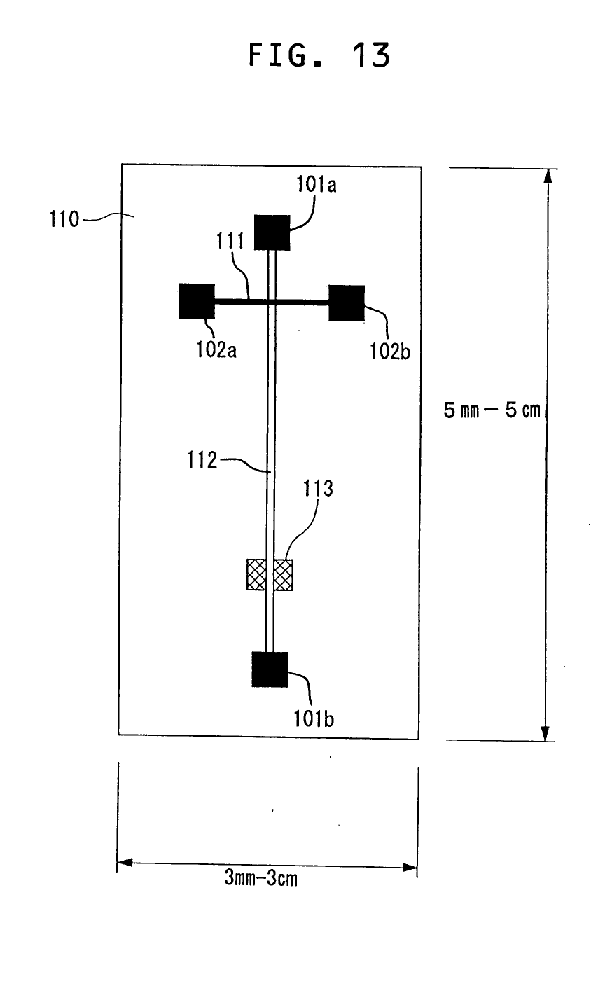

In the following, a method for manufacturing the separation device depicted in FIG. 13 will be described with reference to the drawings. The separation device shown in FIG. 13 is essentially similar to that shown in FIG. 1 except with the collecting channel removed. This separation device is not aimed at classifying separated samples, but used for analyzing components taken out by the detector 113. The separating channel 112 is provided with a sample separating area. The surface of the sample separating area consists of a plurality of hydrophobic areas two-dimensionally arranged at about equally spaced intervals and hydrophilic area that occupy the surface of the sample separating part except for the hydrophobic areas.

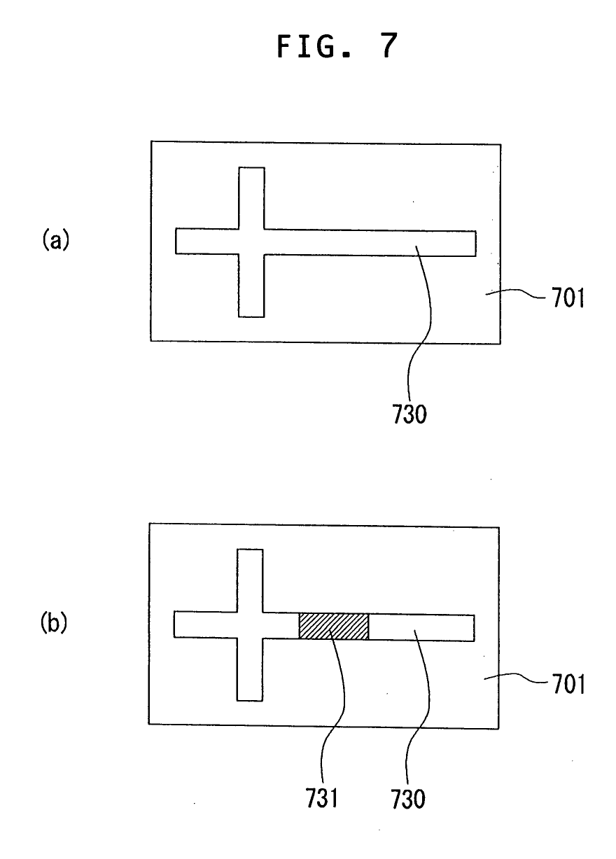

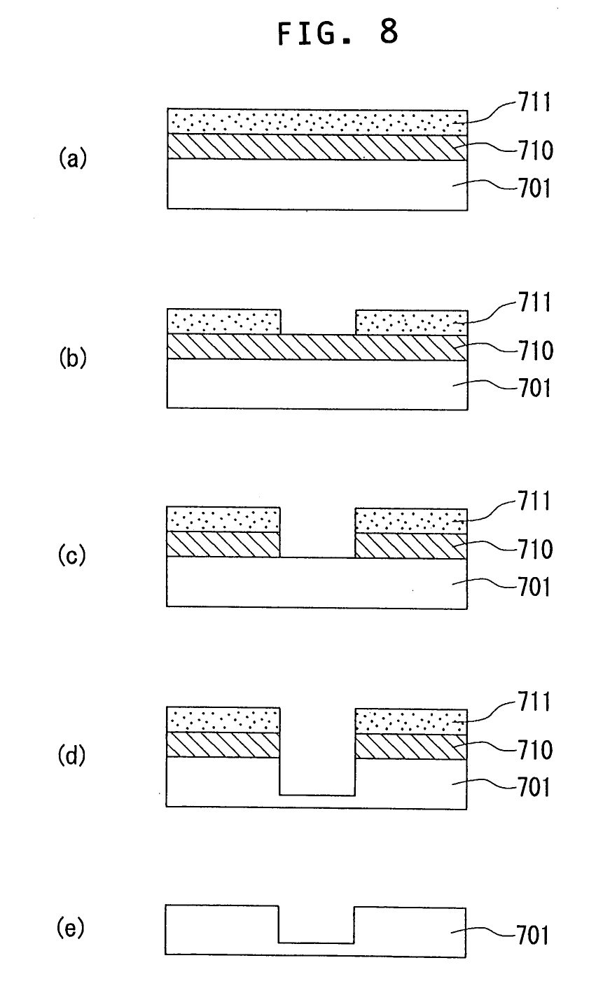

In the process for manufacturing the separation device shown in FIG. 13, a groove 730 is formed first on the surface of the substrate 701 as shown in FIG. 7(a), and then a sample separating area 731 is formed in a prescribed position in the groove 730 as shown in FIG. 7(b). Hereinafter, the process of producing the groove 730 on the substrate 701 in FIG. 7(a) will be described with reference to FIG. 8. Incidentally, in this embodiment, a glass substrate is used as the substrate 701.

First, a hard mask 710 and a resist mask 711 are sequentially formed on the substrate 701 (FIG. 8(a)). Next, a prescribed opening is provided to the resist mask 711 (FIG. 8(b)). After that, dry etching is conducted with the resist mask 711 having the opening, which acts as an etch mask (FIG. 8(c)). SF₆, etc. can be used as etching gas. Subsequently, wet etching is applied to the substrate 701 by using an etchant such as buffered hydrofluoric acid. Generally, the depth of etch is set to about 1 μm. FIG. 8(d) shows the condition after etching is completed. Finally, the hard mask 710 and resist mask 711 are removed (FIG. 8(e)). In this way, the groove 730 as shown in FIG. 7(a) is produced.

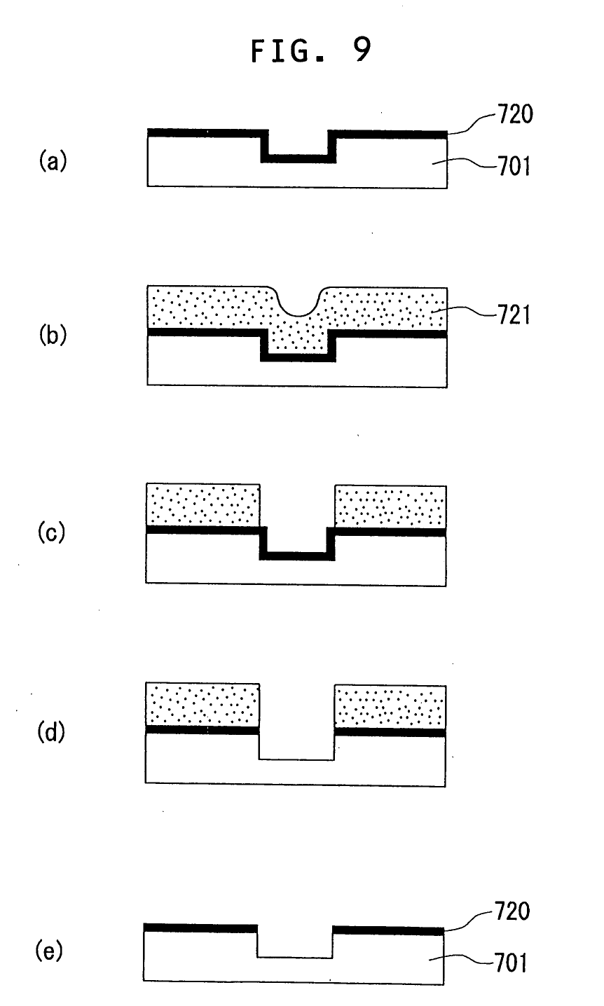

In the process of producing the groove 730 in FIG. 7(a), it is possible to make the surface of the groove 730 hydrophilic, and other parts on the surface of the substrate 701 hydrophobic. Hereinafter, the process of producing such formation will be described with reference to FIG. 9. First, a hydrophobic surface treated layer 720 is formed all over the surface of the substrate 701 in FIG. 8(e) as shown (FIG. 9(a)). Examples of constituent material for the hydrophobic surface treated layer 720 include 3-thiolpropyltriethoxysilane.

Next, the surface of the substrate is coated by a resist 721 by the spin coat method and dried (FIG. 9(b)). Subsequently, an opening is provided to the resist 721 correspondingly to the groove (FIG. 9(c)). Then, dry etching is conducted with the resist 721 having the opening, which acts as an etch mask (FIG. 9(d)). After that, the resist 721 is removed by means of ashing or release agent treatment. In this way, the condition as shown in FIG. 9(e) is achieved. That is, the inner wall of the channel exposes the hydrophilic surface of the substrate 701 which is made of glass material, while other parts are covered by the hydrophobic surface treated layer 720. With this construction, samples are prevented from flowing out of the channel by using a hydrophilic solvent as a carrier solvent.

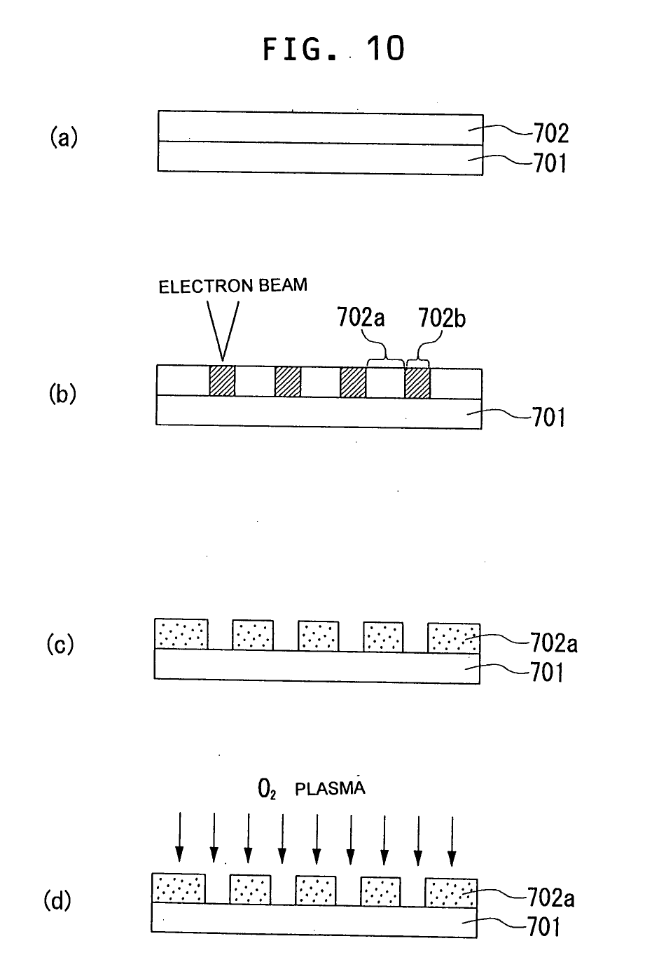

In the following, the process of producing the sample separating area 731 depicted in FIG. 7(b) will be described with reference to FIG. 10. First, an electron beam exposure resist 702 is formed on the substrate 701 as shown in FIG. 10(a). Next, the electron beam exposure resist 702 is exposed to an electron beam to define a prescribed pattern therein (FIG. 10(b)). Exposed parts are dissolved and removed to leave openings in a prescribed pattern as shown in FIG. 10(c). After that, O₂ plasma ashing is conducted as shown in FIG. 10(d). The O₂ plasma ashing is required for patterning in the order of submicron because it activates the surface to which a coupling agent is attached and thus the surface suitable for delicate patterning can be obtained. On the other hand, there is little need for the O₂ plasma ashing when producing a pattern bigger than the order of submicron.

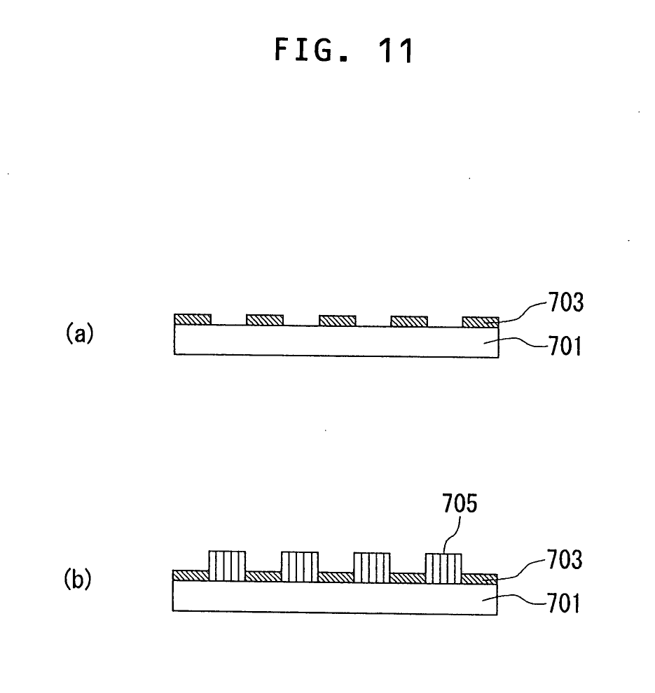

After the completion of the ashing, the condition as shown in FIG. 11(a) is achieved. In FIG. 11(a), the hydrophilic area 703 is formed by the deposition of residual resist and contamination. From this condition, the hydrophobic areas 705 are produced (FIG. 11(b)). In order to form a layer constituting the hydrophobic areas 705, for example, the vapor-phase method can be used. In this case, the substrate and a solution including a coupling agent that contains a hydrophobic group are left in an airtight container for a prescribed period of time to form a layer. According to the method, a treatment layer in a desired pattern can be accurately obtained since a solvent and the like are not attached to the surface of the substrate. Besides, the spin coat method is also applicable to form a layer. Under the spin coat method, the surface of the substrate is treated with a coupling agent solution that contains a hydrophobic group to form the hydrophobic areas 705. 3-thiolpropyltriethoxysilane can be used as the coupling agent that contains a hydrophobic group. Further, the dip method or the like are also used for forming a layer. The hydrophobic areas 705 are not deposited on the hydrophilic area 703 but deposited only on the parts where the substrate 701 is exposed, and accordingly, numbers of the hydrophobic areas 705 are formed at intervals on the surface of the substrate as shown in FIG. 3.

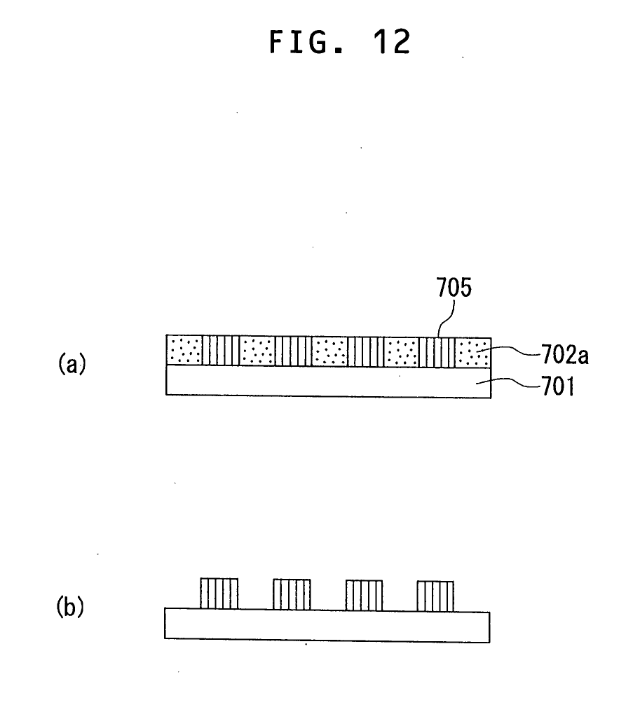

In addition to the aforementioned processes, the following method can be used to obtain the same surface construction as described previously. According to this method, the O₂ plasma ashing is not carried out after forming unexposed parts 702a in a pattern as shown in FIG. 10(c), but 3-thiolpropyltriethoxysilane is deposited in each opening of the resist to form the hydrophobic areas 705. After that, wet etching is conducted with a solvent capable of selectively removing the unexposed parts 702a to achieve the condition as shown in FIG. 12(b). On this occasion, it is important to select a solvent which does not damage a layer that forms the hydrophobic areas 705. Examples of such solvent include acetone.

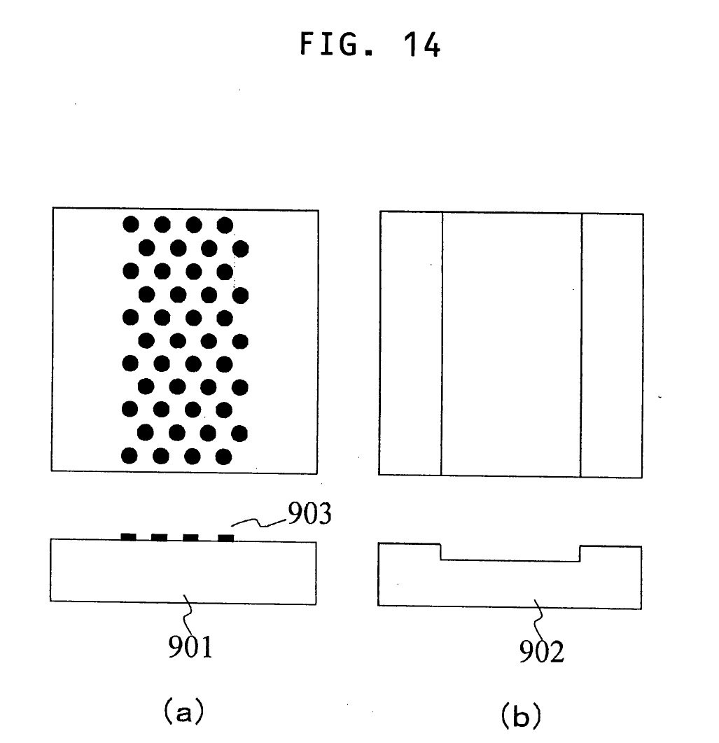

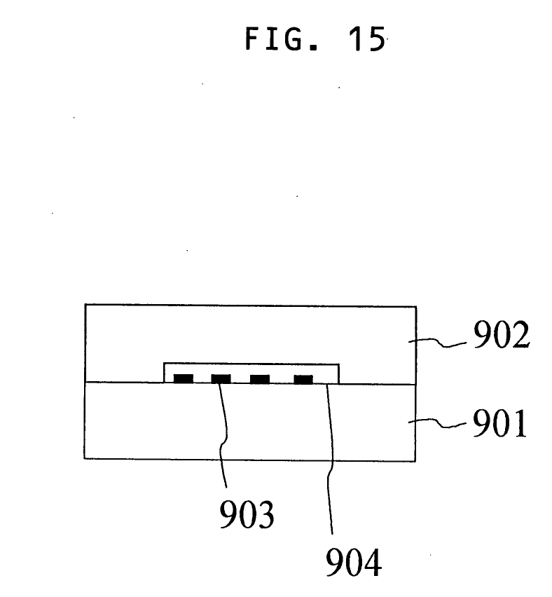

While the hydrophobic areas are formed in the channel in the above-described embodiment, the following method is also applicable. In this method, two types of substrates as shown in FIG. 14(a) and FIG. 14(b) are used. In FIG. 14(a), a glass substrate 901 has hydrophobic layers 903 including a compound that contains a hydrophobic group such as 3-thiolpropyltriethoxysilane thereon. The hydrophobic layers 903 are defined in a prescribed pattern. The position in which the hydrophobic layers 903 are provided forms the sample separating part. On the other hand, in FIG. 14(b), a glass substrate 902 has a stripe groove on its surface. The groove serves as a channel for samples. The hydrophobic layers 903 are formed by following the process described previously. The stripe groove on the surface of the glass substrate 902 can be easily produced by wet etching using an etch mask in the same manner as above stated. The sample separation device of the present invention can be obtained by bonding the two substrates together as shown in FIG. 15. A space 904 between the two substrates serves as a channel for samples. According to this method, the hydrophobic layers 903 are formed on a flat surface, which facilitates the manufacture of the separation device, thus ensuring excellent manufacturability.

A micropattern of hydrophilic/hydrophobic areas may be produced by forming a layer of a silane coupling agent all over the surface of the substrate by the LB membrane lifting method as described in “Nature, vol. 403, 13 Jan. 2000.

Additionally, the channel itself can be formed through the hydrophobic/hydrophilic treatment.

In the case of producing the channel by the hydrophobic treatment, a hydrophilic substrate such as a glass substrate is used, and parts which serve as walls of the channel are made hydrophobic areas. Water flows around the hydrophobic areas, thus forming the channel.

The channel may be covered as well as uncovered. When providing a cover to the channel, it is required to leave a space of some g m between the cover and the substrate. The space can be formed by bonding an edge of the cover to the substrate by using a viscous resin such as PDMS (polydimethylsiloxane) or PMMA (polymethylmethacrylate) as an adhesive. Even as the cover is bonded to the substrate only at the near edge, water avoids the hydrophobic areas and feeds into the part that forms the channel. Thereby, the channel is formed.

On the other hand, when producing the channel by the hydrophilic treatment, a hydrophobic substrate or a substrate which has been made hydrophobic through the silazane treatment, etc. is used. The channel having hydrophilic areas is produced by applying the hydrophilic treatment to the part to be occupied by the channel. Water feeds into only the hydrophilic areas, and the channel is formed.

The hydrophobic/hydrophilic treatment can be conducted with the use of printing techniques such as stamping and ink-jet printing.

For example, PDMS resin is used for stamping. Since PDMS resin is obtained by polymerizing silicone oil, even after the product have been resinified to be PDMS resin, spaces between its molecules are filled with silicone oil. Accordingly, when bringing PDMS resin in touch with a hydrophilic surface such as a glass surface, parts of the glass surface, which have touched PDMS resin, become extremely hydrophobic and repel water. On this account, by forming a concavity on a PDMS block in alignment with the part to be occupied by the channel and stamping the block on a hydrophilic substrate, the channel can be easily produced through the hydrophobic treatment.

Such treatment can be conducted by an ink-jet printer and the like.

In the case of using the ink-jet printer, low-viscosity silicone oil is used as printing ink for the ink-jet printer and a thin layer of hydrophilic resin such as a sheet of polyethylene, PET (polyethylene terephthalate) or acetylcellulose (cellophane) is used as a printing paper. By printing a pattern in a manner such that silicone oil is applied to the part to be walls of the channel, the walls are made hydrophilic.

Additionally, it is possible to provide a filter, which allows substances smaller than a particular size to pass through and catches substances larger than the size, in the channel by the use of hydrophobic surface-treated patches (hydrophobic patches) or hydrophilic surface-treated patches (hydrophilic patches).

For example, in the case of forming a filter with hydrophobic patches, the patches are arranged linearly at regular intervals so as to define a broken line pattern in the filter. The distance between two adjacent patches is set larger than the size of a substance intended to be passed and smaller than the size of a substance intended to be caught. In order to remove substances larger than 100 μm, the distance between two adjacent hydrophobic patches has to be narrower than 100 μm, and set to, for example, 50 μm.

The filter can be obtained by integrally forming a hydrophobic area pattern for constituting the channel and the broken line pattern made by the hydrophobic patches. Examples of methods for producing the filter include photolithography and SAM layer-forming, stamping, and ink-jet printing.

Incidentally, in the case of forming the filter in the channel, the filter surface can be arranged at right angles or in parallel with a flow. It is preferable that the filter surface is arranged in parallel with a flow for causing less clogging and allowing a larger space for the filter as compared to the case where it is arranged at right angles to a flow. In this case, the width of the channel is expanded (e.g. 1000 μm), and 50 μm by 50 μm square hydrophobic patches are formed or arranged in the center part of the channel at intervals of 50 μm in the direction of flow, in such a manner as to divide the channel lengthwise into two parts. When feeding a liquid containing substances to be separated from one side of the divided channel, substances larger than 50 μm are removed by filtration from the liquid and the filtrate is obtained from the other side of the channel.

Hereinafter a description will be give of a practical example of the present invention, however, this is not intended to be limiting of the invention.

### EXAMPLE

A channel was produced by way of trial for separating cell-sized substances according to size.

The size of cells ranges from 10 μm to 1 μm. Specifically, a red blood cell is of a size of about φ 7.5 μm, a white blood cell is of a size of about φ 10 μm, a blood platelet is of a size of about φ 2 μm and, on the other hand, bacteria is of a size of about φ 1 μm. Therefore, fluorescent beads (Polysciences, Inc. Fluoresbright Carboxylate (2.5% Solid-Latex)) of two different sizes, φ 1 μm and φ 10 μm, were used as separation targets. Observation of these beads showed that:

(1) air bubbles were formed on hydrophobic surface-treated patches (hydrophobic patches);

(2) the beads could not enter into the air bubbles on the hydrophobic patches, and the air bubbles on the patches functioned as obstacles in the channel;

(3) moving speed of the beads in both sizes were reduced at a touch with the air bubbles on the hydrophobic patches; and

(4) the beads of a size of φ 10 μm were more strongly influenced by the touch.

The separation channel was produced by the following procedure.

A separation area 10 mm wide by 50 mm long was formed longitudinally in the vicinity of the center of a cover glass for microscope 24 mm by 50 mm. Subsequently, 100 μm by 100 μm square hydrophobic patches were arranged with a distance of 200 μm between each of them so as to define a tetragonal lattice pattern in the entire separation area.

In order to produce the hydrophobic patches, negative photoresist (S1818) overlaid on the cover glass was exposed to light through photolithography to create a pattern of square patches, and square portions of the resist were removed. After applying oxygen plasma ashing (350 W, 0.5 Torr, 10 minutes), hydrophobic silazane membranes (SAM) were formed on the respective portions where glass surface was exposed by treating the surface with silazane vapour. Then, the resist was removed by acetone.

By this means, the hydrophobic patches were formed on two cover glasses. One of them was attached on a glass slide of 10 cm by 10 cm with an instant adhesive, and a polyethylene sheet 18 μm thick was put on the parts other than the separation area. After that, the other cover glass was set thereon so that the treated surfaces of both the cover glasses faced each other to produce the separation channel.

1×TBE buffer was added by a pipette to one end of the separation channel 10 mm wide by 50 mm long by 18 μm deep formed through the above procedure. 1×TBE buffer automatically filled the separation channel due to the capillary phenomenon.

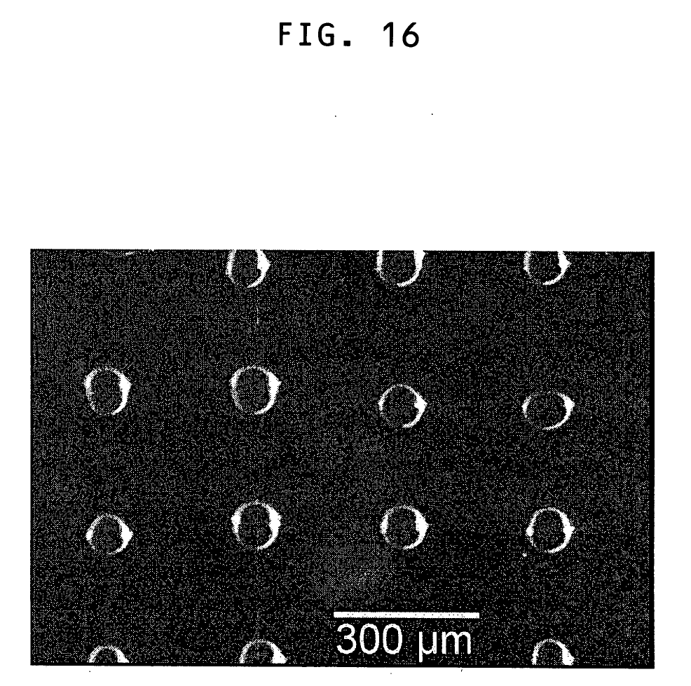

FIG. 16 shows a pattern of air bubbles formed after adding TBE buffer. In FIG. 16, it can be observed that round air bubbles were formed in places marked by the hydrophobic patches, and that the air bubbles formed air columns on the surface of the separation channel. The distance between two adjacent air bubbles was 300 μm.

The two types of beads were diluted with or suspended in 1×TBE buffer in concentrations appropriate for observation. Then, 0.5 μl of the bead suspension was added to one end of the separation channel filled with 1×TBE buffer. The bead solution entered into the channel due to the capillary phenomenon, and stopped. The two types of beads could be clearly distinguished by a difference in size under the microscope.

Next, 200 μl of 1×TBE buffer was added to the same end of the separation channel at a time. The added buffer automatically entered into the channel, and spilled out of the other end of the channel. In this process, the bead suspension which had stayed at the end of the channel was swept through the separation channel. Aspects of the flow was observed by a CCD camera.

Since the beads of a size of φ10 μm sink to the bottom of the channel when too much time has passed after the addition of TBE buffer, the observation was carried out for 3 seconds after the addition while the beads were yet floating

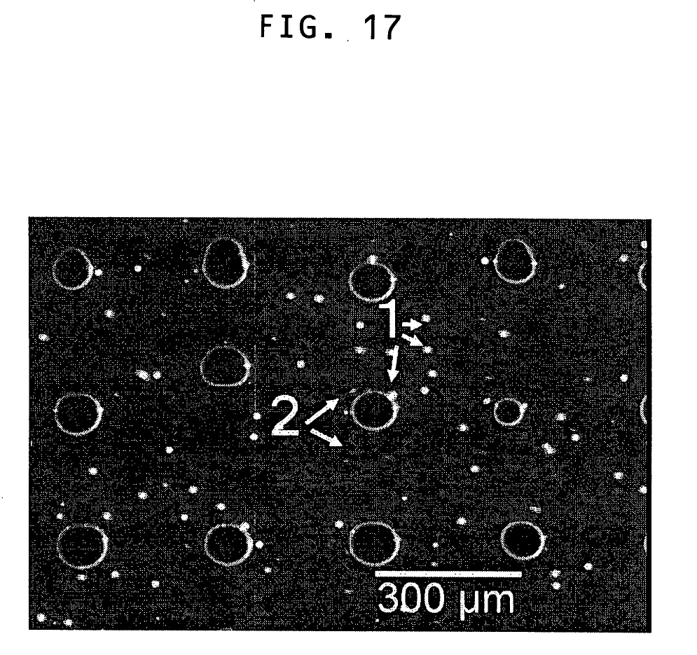

FIG. 17 shows the beads in collision with bubbles. The beads flowed from the right to the left as seen in the drawing at a current velocity of 300 μm/s. The two types of beads moved with the flow at approximately identical velocities everywhere but at the air bubbles, temporarily stopped in collision with the air bubbles on the hydrophobic patches. After that, although the beads skirted around the air bubbles and resumed flowing, their moving velocity was reduced to about one-third. This means that the hydrophobic patches and the air bubbles thereon constitute a limiting factor of the movement of the beads. Apparently, the beads of a size of φ10 μm (denoted by the numeral 1 in FIG. 17) skirted around the air bubbles with a lower velocity as compared to the smaller beads (denoted by the numeral 2 in FIG. 17, appearing as streaks because of shutter speed), and the velocity was about two-thirds of that of the smaller beads. This suggests that the size of the bead makes a difference in velocity at which the bead skirts around the air bubble by contact with the hydrophobic patch. Thus, a separating effect is achieved. By defining a dense pattern of the hydrophobic patches, the frequency of bead collision can be increased, and the separating effect can be expected to become even more prominent.

### INDUSTRIAL APPLICABILITY

As set forth hereinabove, in accordance with the present invention, a pattern of hydrophilic/hydrophobic areas is defined in the surface of the sample separating part, and separation is carried out due to the surface characteristics. That is, the hydrophilic/hydrophobic areas arranged at intervals on the surface of the sample separating part serve as a sieve, thus enabling the efficient separation of target components. Since the separation of samples is carried out according to size and polarity, it is possible to realize separative power higher than ever. Besides, the sample separating part is formed by surface treatment, thus providing excellent manufactural stability. Moreover, separation is carried out due to the surface characteristics and therefore requires shorter period of time and a less amount of samples. Furthermore, the separation device of the present invention has few clogging problems, and can be cleaned very easily by flushing the surface of the sample separating part with cleaning solution. Thus, it is possible to realize both high-precision separation and excellent operationality.

1-19: (canceled).

20. A separation device comprising at least one substrate and a channel formed on the surface of the substrate for a flow of samples wherein the channel comprises two kinds of areas, one being lyophilic and the other being lyophobic.

21. The separation device claimed in claim 1, wherein the lyophilic area and/or the lyophobic area comprises a layer having a hydrophobic group or a layer having a hydrophilic group formed on the surface of the substrate.

22. The separation device claimed in claim 1, further comprising a sample feeding part, a sample waste collection part, and a sample separating part in between the sample feeding part and sample waste collection part, wherein the surface of the sample separating part includes a plurality of first areas and second area, one being lyophobic and the other being lyophilic.

23. The separation device claimed in claim 3, wherein the lyophilic area and/or the lyophobic area comprises a layer having a hydrophobic group or a layer having a hydrophilic group formed on the surface of the substrate.

24. The separation device claimed in claim 3, wherein the first areas are two-dimensionally arranged at about equally spaced intervals, and the second area occupies the surface of the sample separating part except for the first areas.

25. The separation device claimed in claim 5, wherein the lyophilic area and/or the lyophobic area comprises a layer having a hydrophobic group or a layer having a hydrophilic group formed on the surface of the substrate.

26. The separation device claimed in claim 5, wherein the channel is covered by a cap, and the lyophilic area and/or the lyophobic area are formed on the surface of the cap.

27. The separation device claimed in claim 3, further comprising a plurality of sample separating parts, wherein the distance between two adjacent sample separating parts is wider than the space between the respective first areas which form each sample separating parts, and the space between the respective first areas varies from one separating part to another.

28. The separation device claimed in claim 8, wherein the lyophilic area and/or the lyophobic area comprises a layer having a hydrophobic group or a layer having a hydrophilic group formed on the surface of the substrate.

29. The separation device claimed in claim 8, wherein the channel is covered by a cap, and the lyophilic area and/or the lyophobic area are formed on the surface of the cap.

30. The separation device claimed in claim 1, wherein at least part of the channel for a flow of samples is a space or a clearance gap formed between two adjacent substrates.

31. The separation device claimed in claim 2, wherein at least part of the channel for a flow of samples is a space or a clearance gap formed between two adjacent substrates.

32. The separation device claimed in claim 3, wherein at least part of the channel for a flow of samples is a space or a clearance gap formed between two adjacent substrates.

33. The separation device claimed in claim 4, wherein at least part of the channel for a flow of samples is a space or a clearance gap formed between two adjacent substrates.

34. The separation device claimed in claim 5, wherein at least part of the channel for a flow of samples is a space or a clearance gap formed between two adjacent substrates.

35. The separation device claimed in claim 6, wherein at least part of the channel for a flow of samples is a space or a clearance gap formed between two adjacent substrates.

36. The separation device claimed in claim 8, wherein at least part of the channel for a flow of samples is a space or a clearance gap formed between two adjacent substrates.

37: The separation device claimed in claim 9, wherein at least part of the channel for a flow of samples is a space or a clearance gap formed between two adjacent substrates.

38. A fluidic device comprising at least one substrate and a channel formed on the surface of the substrate for a flow of samples, wherein at least part of the channel includes a first area being lyophilic and a second area being lyophobic.

39. The fluidic device claimed in claim 19, wherein each of the substrates is arranged parallel to the surface of another substrate, on which the first area and the second area have been formed, so as to leave a space therebetween.

40. A method for manufacturing a fluidic device comprising at least one substrate and a channel formed on the surface of the substrate for a flow of samples, comprising the steps of:
after making a mask having openings on at least part of the surface of the channel, fixing a chemical compound having a lyophobic group from the openings to the surface of the substrate in the case where the surface of the substrate is lyophilic, or fixing a chemical compound having a lyophilic group from the openings to the surface of the substrate in the case where the surface of the substrate is lyophobic; and
removing the mask so as to form sample separating parts each including a plurality of lyophobic areas or lyophilic areas.

41. The method for manufacturing a fluidic device claimed in claim 21, further comprising the step of:
arranging each of the substrates parallel to the surface of another substrate, where the lyophobic compound or the lyophilic compound has been fixed, so as to leave a space or a clearance gap therebetween.

42, A method for manufacturing a fluidic device comprising at least one substrate and a channel formed on the surface of the substrate for a flow of samples by using a printing technology including stamp or ink jet printing, comprising the step of:
fixing a chemical compound having a lyophobic group to the surface of the substrate in the case where the surface of the substrate is lyophilic, or fixing a chemical compound having a lyophilic group to the surface of the substrate in the case where the surface of the substrate is lyophobic so as to form sample separating parts each including a plurality of lyophobic areas or lyophilic areas.

43. The method for manufacturing a fluidic device claimed in claim 23, further comprising the step of:
arranging each of the substrates parallel to the surface of another substrate, where the lyophobic compound or the lyophilic compound has been fixed, so as to leave a space or a clearance gap therebetween.

44. A method for manufacturing a separation device comprising at least one substrate and a channel formed on the surface of the substrate for a flow of samples, comprising the steps of:
cutting a groove on the surface of the substrate to form the channel;
after making a mask having openings on at least part of the surface of the channel, fixing a chemical compound having a lyophobic group from the openings to the surface of the substrate in the case where the surface of the substrate is lyophilic, or fixing a chemical compound having a lyophilic group from the openings to the surface of the substrate in the case where the surface of the substrate is lyophobic; and
removing the mask so as to form sample separating parts each including a plurality of lyophobic areas or lyophilic areas.

45. A method for manufacturing a separation device comprising a substrate, a cap, a channel formed on the surface of the substrate for a flow of samples, and sample separating parts provided in the channel, comprising the steps of:
cutting a groove on the surface of the substrate to form the channel; after making a mask having openings on at least part of the surface of the cap, fixing a chemical compound having a lyophobic group from the openings to the surface of the cap in the case where the surface of the cap is lyophilic, or fixing a chemical compound having a lyophilic group from the openings to the surface of the cap in the case where the surface of the cap is lyophobic;
removing the mask so as to form the sample separating parts each including a plurality of lyophobic areas or lyophilic areas; and
stacking the cap on the substrate so that the sample separating parts are exposed at least on a part of the channel.

46. A method for manufacturing a separation device comprising at least one substrate, a channel formed on the surface of the substrate for a flow of samples, and sample separating parts provided in the channel, comprising the steps of:
cutting a groove on the surface of the substrate to form the channel; and
fixing a chemical compound having a lyophobic group to at least part of the surface of the channel in the case where the surface of the substrate is lyophilic, or fixing a chemical compound having a lyophilic group to at least part of the surface of the channel in the case where the surface of the substrate is lyophobic so as to form the sample separating parts each including a plurality of lyophobic areas or lyophilic areas, by using a printing technology including stamp or ink jet printing.

47. A method for manufacturing a separation device comprising a substrate, a cap, a channel formed on the surface of the substrate for a flow of samples, and sample separating parts provided in the channel, comprising the steps of:
fixing a chemical compound having a lyophobic group to at least part of the surface of the cap in the case where the surface of the cap is lyophilic, or fixing a chemical compound having a lyophilic group to at least part of the surface of the cap in the case where the surface of the cap is lyophobic so as to form the sample separating parts each including a plurality of lyophobic areas or lyophilic areas, by using a printing technology including stamp or ink jet printing; and
stacking the cap on the substrate so that the sample separating parts are expose
